(19) United States
(12) Patent Application Publication
(10) Pub. No.: US 2009/0017487 A1
(43) Pub. Date: Jan. 15, 2009

(54) BIOGAS PRODUCTION FROM BMR PLANTS

(75) Inventor: Peter Schlotter, Muenchen (DE)

Correspondence Address: DOW AGROSCIENCES LLC, 9330 ZIONSVILLE RD, INDIANAPOLIS, IN, 46268, US

(73) Assignee: Dow AgroSciences LLC, Indianapolis, IN (US)

(21) Appl. No.: 12/131,282

(22) Filed: Jun. 2, 2008

Related U.S. Application Data
(60) Provisional application No. 60/932,461, filed on May 31, 2007.

(51) Int. Cl. C12Q 1/02; C12P 1/04; C12P 5/02

(52) U.S. Cl. 435/29; 435/170; 435/167

(57) ABSTRACT

Methods and compositions to produce biogas and fertilizer from plant material that have reduced lignin content relative to a wild-type plant material, for example, a brown midrib (BMR) plant, are disclosed. Methane yields from BMR plants including BMR corn are significantly higher, thereby increasing the efficiency of methane production. Anaerobic digestion of plant material derived from BMR plants results in an increased methane yield.

### BACKGROUND

Increasing global energy requirements in an environmentally friendlier fashion has resulted in increasing focus on renewable energy sources. In contrast to energy obtained from fossil fuels that release additional CO₂ into the atmosphere, energy production from biomass is regarded as being “CO₂-neutral”, since the amount of CO₂ released by combustion of a given amount of the biomass corresponds to the amount of CO₂ which was originally taken up from the atmosphere during the build-up of that biomass.

Among the fuels that are derived from plant biomass, ethanol has received considerable attention as a potential replacement for or as a supplement to petroleum-derived liquid hydrocarbon products. This ethanol, also called as “bioethanol” has been used for a variety of other purposes. Materials such as straw, maize stems, forestry waste (log slash, bark, small branches, twigs and the like), sawdust and wood-chips are all materials that have been used to produce bioethanol.

Biogas, which generally relates to methane and CO₂, has been produced from a variety of sources such as, animal wastes (manures), agricultural wastes, industrial wastes, municipal wastes, and sewage wastes. In many instances, biogas generation from such wastes often involve anaerobic digestion or fermentation of the biological material in the presence of suitable microbes. Some of these biogas generation processes involve certain pretreatments, such as, for example, treating a polysaccharide material such as cellulose, hemicellulose and lignocellulose by acid hydrolysis.

Microbial conversion of organic matter to methane has become an attractive option for treatment of wastes for energy recovery. Some of the characteristic features of anaerobic digestion with microbes are: (i) bacteria involved in biogas production process are anaerobes and slow growing; (ii) some degree of metabolic specialization is observed in these anaerobic microorganisms; and (iii) that most of the free energy present in the substrate is captured in the terminal product methane. Because of the slow growth and low energy requirement for these microbes, less microbial biomass is produced and therefore, disposal of sludge after the digestion may not be an issue.

A variety of plant feed materials have been used as animal feed material. For example, a typical animal feed derived from cultivated plants include plant material from corn and sorghum. Brown midrib (BMR) corn plants exhibit a reddish-brown pigmentation of the leaf midrib. The pigmentation is also seen in rind and pith. Coloring eventually disappears on leaves, but remains in the stalk. The brown midrib phenomenon is also found in sorghum and some varieties of millet.

In the past, generally, the cost basis of bioethanol and that of biogas have not been competitive with that of traditional fossil fuels. However, with the supply of fossil fuels decreasing with an increasing global demand for fossil fuels, production of renewable fuels from biomass has become an attractive and viable option. Nevertheless, the efficiency of energy recovery from biomass needs to be increased by reducing production costs, fermentation time, while increasing the yield per unit weight of biomass. One method to increase the efficiency of biogas generation is to find a suitable plant material with an increased methane yield potential.

### SUMMARY

Methods to produce biogas from a plant variety/hybrid that has reduced lignin content relative to a wild-type plant are disclosed. Suitable plant material includes brown midrib (BMR) plants that have reduced lignin content. Methods to produce biogas includes the steps of obtaining a substrate from the BMR plant and anaerobically digesting the substrate in the presence of a microbial inoculum to produce biogas. Suitable BMR plants include BMR corn or BMR sorghum. The biogas produced contains a significant amount of methane. The anaerobic digestion may be performed in an anaerobic reactor (digester).

In an embodiment, methane gas is produced at a concentration of about 340-350 liters of methane per kilogram of dry organic matter of the plant material and the biogas contains about 50% methane by volume.

In an embodiment, the microbial inoculum includes acid forming bacteria and methane producing bacteria. The microbial inoculum may also include thermophilic and mesophilic bacteria.

Suitable plant substrate material includes silage, stover, hay, feed concentrate, and any other plant part that contains organic matter capable of producing biogas upon treatment with microbes.

In an embodiment, the digestion is performed at a temperature of about 55° F. to about 100° F. and at a pH of about 7-8.

In an embodiment, the plant substrate is processed prior to the digestion and the processing steps include drying, milling, and acid hydrolysis.

A method for producing a digested substrate, e.g., fertilizer, from a plant material that has reduced lignin content compared to a wild-type plant material, the method includes the steps of obtaining a substrate from the plant; anaerobically digesting the substrate in the presence of a microbial inoculum to produce biogas; and recovering the remaining digested substrate as the fertilizer. The fertilizer or the digested susbtrate may be in a dried state or as a sludge. The plant material may be from a BMR plant.

A method for determining a methane yield potential of a plant with reduced lignin content, the method includes the steps of:

(a) obtaining a substrate sample from the plant;

(b) treating the substrate sample and a microbial inoculum in a container such as, for example a fermentor, so that the substrate sample is placed in contact with the microbial inoculum;

(c) providing conditions for the production of methane and/or biogas from the substrate sample; and

(d) determining the methane or biogas yield potential of the plant variety/hybrid.

A method for increasing the efficiency of biogas generation, the method includes the steps of:

(a) obtaining a BMR plant or a plant material with reduced lignin content, wherein a methane yield potential is more than 300 liters of methane per kilogram of dry organic matter of the plant; and

(b) generating biogas from the BMR plant or the plant with reduced lignin content thereby increasing the efficiency of biogas generation.

In an embodiment, the efficiency of biogas generation is increased by decreasing the retention time and increasing the methane yield per unit dry weight of the organic matter.

### BRIEF DESCRIPTION OF THE DRAWINGS

FIG. 1 is a schematic illustration of an anaerobic digester with a few exemplary purification systems.

FIG. 2 shows progression of methane production from the inoculum, substrate, hay and concentrate feed.

FIG. 3 shows the evolution of the methane production of three replicates from two selected maize variants/hybrids.

FIG. 4 is a bar graph showing methane production per kg. of organic dry matter for BMR and non-BMR maize plants.

### DETAILED DESCRIPTION

The use of cultivated biomass as a feeding material in biogas plants is an attractive option for renewable energy generation. Cultivated plant material has traditionally been used for animal feed purposes. However, the quality and the characteristics of the available plant material that are used for animal feed and biogas generators are different. Data provided herein demonstrate that methane yields and methane content in the biogas generated by plant material with reduced lignin content, e.g., brown midrib (BMR) plants, is significantly higher than non-BMR plants (plant material with higher lignin content), on an average. Any plant material that has reduced lignin content is suitable for biogas production utilizing the methods disclosed herein.

In an embodiment, Hohenheim Biogas yield Test developed at the University of Hohenheim and as described in the Germany VDI 4630 standards guidance was applied to determine the methane yield potential of several BMR plant varieties.

In an embodiment, plant substrate including silage from BMR corn plants were used. Suitable BMR plants include BMR corn/maize varieties, for example, F2F485, F2F610, F2F633, F2F699, F2F721 and 2F797.

Suitable plant materials also include BMR sorghum and BMR millet. The plant substrate material, such as straw, may be further processed by reducing to a particle size of about 0.2 mm to about 2.00 mm to enhance biogas generation. Pretreatment with acid or alkali may also increase biogas production, depending the plant material used.

In an embodiment, the plant substrate material consists essentially of a plant substrate derived from a BMR plant. The ratio of BMR plant material to non-BMR plant material can range from about 1:1 to about 10:1; 2:1 to about 5:1, and 3:1 to about 7.5: 2.5. The plant material may contain at least 95% BMR plant-derived material, or 90% BMR plant-derived material, 85% BMR plant-derived material and 75% BMR plant-derived material.

In an embodiment, methane gas is produced at a concentration of about 340-350 liters of methane per kilogram of dry organic matter of the plant material and the biogas contains about 50% methane by volume. In another embodiment, methane gas is produced at a concentration about 342 liters to about 355 liters of methane per kilogram of dry organic matter of the plant material. In another embodiment, methane gas is produced at a concentration of no less than about 340 liters of methane per kilogram of dry organic matter of the plant material. In another embodiment, methane gas is produced at a concentration of at least 345-350 liters of methane per kilogram of dry organic matter of the plant material. The methane content may range from about 45% to 50%, 48% to 51%, and 50% to 55% of the total biogas volume. Methane content may also be up to 65% to 70% of the total biogas volume.

A variety of digesters are suitable for anaerobic production of biogas. Suitable digesters include plug-flow digesters, mix and hybrid digesters, fixed film digester, upright cylinder digester, solid state stratified bed (SSB) digester, stirred tank reactor (STR), and batch reactors.

Based on the biogas yield potential of the plant material used, volume or amount of the plant material and the inoculum characteristics, the size of the reactor is calculated. Design characteristics of a biogas reactor based on anaerobic digestion are commercially available and are known to skilled artisans.

Anaerobic digestion refers to a process whereby organic waste is broken down in a controlled, oxygen free environment by microbes, converting a substantial portion of the stored carbon into methane.

Biofuel refers to any fuel generated from a biological material, such as, for example, biogas, bioethanol, and others.

Biogas generally refers to gaseous components generated from biological material. Biogas includes methane, carbon dioxide and other byproducts such as hydrogen sulfide. Methane constitutes a significant portion of the biogas as a combustible energy source.

Biomass or biosubstrate generally refers to any biological material capable of being used for generation of biofuels.

BMR refers to brown midrib varieties of plants including corn/maize, sorghum and millet. BMR mutants include both naturally occurring and those that have been selected using classical marker assisted breeding techniques and genetic engineering techniques. BMR also refers to BMR mutants, hybrids, those plants that have been crossed to BMR plants and variants thereof. BMR plants may also have one or more BMR mutations that result in a BMR phenotype or genotype. BMR phenotypes may have reduced lignin and elevated cellulose per unit weight.

Concentrate feed refers to a substrate including condensed, optimized and enriched feed substrate derived from grain, soybean, nutrients and other plant material that may have been processed.

Efficiency of methane production generally relates to an economical method of generating methane gas from organic substrates by improving one or more of the following variables: quality of biomass, reduced retention time of the substrate in the digester, increased methane yield per unit biomass, increased methane content per unit volume of biogas, and other methods that reduce the overall cost of methane production.

Fertilizer refers to a solid or semi-solid byproduct of anaerobic digestion of plant material that contains one or more nutrients to support plant growth.

Microbial inoculum refers to one or more microbial isolates in a sufficient concentration to anaerobically digest a plant-derived substrate and produce methane gas. A representative microbial inoculum contains both acid forming bacteria and methanogenic bacteria. The microbial isolates in the inoculum may be obtained from a cultured source (e.g., bioreactor) or from a natural source (preexisting soil, decomposing material or semi-liquid manure). The inoculum may also contain genetically engineered microorganisms to perform a specific function, for example, expressing recombinant cellulases to break-down cellulose. Depending upon the temperature used, mesophilic and thermophilic bacteria are included in the inoculum.

Plant substrate or plant material refers to plant derived substance, e.g. silage from maize or other crops. The plant substrate or plant material can also be processed by grinding or milling to obtain a coarse particle size of about 0.2 mm to about 2.0 mm. Silage derived from BMR plants is also used as a substrate for the methane/biogas production.

Stover includes the leaves and stalks of corn (maize), sorghum or soybean plants that are generally left in a field after harvest.

Applicants have made a deposit of at least 2,500 seeds of hybrid corn plant DAS05401 with the American Type Culture Collection (ATCC), Manassas, Va. 20110 USA, under ATCC Accession No. ______. The seeds deposited with the ATCC on ______ were taken from a deposit maintained by Agrigenetics, Inc. d/b/a Mycogen Seeds since prior to the filing date of this application. Access to this deposit will be available during the pendency of the application to the Commissioner of Patents and Trademarks and persons determined by the Commissioner to be entitled thereto upon request. Upon allowance of any claims in the application, the Applicant(s) will maintain and will make this deposit available to the public pursuant to the Budapest Treaty.

### EXAMPLES

The following examples are for illustrative purposes only and are not intended to limit the scope of this disclosure.

### Example 1

Measurement of biogas yield potential for plant material substrates. To design a biogas generator where biomass, including plant materials is anaerobically digested to produce biogas, the biogas yield potential of the plant substrate may be measured. One such biogas yield test is described in a published report by Helfrich & Oechsner (2003), The Hohenheim biogas yield test, Landtechnik, 58: 148-149.

Briefly, the Hohenheim biogas yield test method is a non-continuous (batch) digestion process involving a single feeding of the substrate at the start of an experiment. The batch reactor (digester) is a small scale, 100-ml, calibrated glass syringe with a gas outlet. The syringe's plug is sealed against the glass syringe by means of an non-biodegradable lubricant. The bored side of the syringe includes a connected hermetic pipe, which is closed by a fastening clip. The generated biogas is let out of the syringe for measurement of the methane content. Depending on the size of the equipment, 60 to 200 syringes can fit inside a motorized rotating support. The rotation of the rotor ensures adequate mixing of the substrate. The rotating unit is built inside a thermostat regulated incubator, where the digesters filled with substrates and the gas storage unit are heated to desired temperatures.

In an embodiment, a single batch or load of the test substrate is added to the inoculum fluid. This aqueous inoculum solution contains biogas producing strains of microorganisms, and provides the required pH conditions for the anaerobic digestion. The inoculum was produced from a 400 L reactor, and after withdrawal from the digester, the inoculum was kept away from air contamination and maintained at the digestion temperature. Coarse particles were removed from the inoculum by sieving.

The substrate amount for determining the methane yield potential was less than one gram. The plant substrates samples were dried for 48 hours in a drying chamber at 50 to 60° C. Then, the plant substrate particles sizes were reduced to 1 mm using a cutting mill. The sample drying and milling procedures ensured homogeneity and a reasonable representative digestion sample from the 400 g crop substrate sample for effective digestion. The combination of drying and milling also corresponded to the usual preparation stages for studying feed materials.

The total solids (TS) and volatile solids (VS) contents were determined from the crop samples, to provide a reference basis to express the values of the methane yields. To initiate the reaction, 30 mL of inoculum was added to the syringe digester, and then 400 mg of crop substrate was measured by a precision weighing device. Variations up to 1% of the weight value, if present, were detected and documented. To avoid overloading of the process with the increased crop substrates, adequate inoculum doses were applied. However, caution was exercised to ensure that a substantial portion of the biogas generated was from the crop substrate and not the inoculum, to minimize any influence on biogas production from the inoculum itself. Depending on the substrate's energy content, the optimal dry matter ratio between inoculum and test substrate lies between 2 and 3.1.

After weighing the inoculum and the test substrate, the syringes are closed with the plugs and brought inside the rotating support. The gas production normally starts directly after filling the syringes. The measurements of the gas volume on the graduated scale of the syringe were done at short intervals of time at the beginning of the experiment. The measurement intervals became gradually longer as the experiment progressed. After opening the fastening clip of the exhaust pipe, the gas was let out of the syringe and analyzed for its methane content using any standard methodology, for example, gas chromatography (GC). The remaining volume, after the gas removed from the syringe, was also measured. The experiment may be stopped on the day that the expected remaining gas production is lower than 1% of the total amount of gas already released.

The gas values were converted to normalized conditions upon consideration of temperature, gas pressure and water vapor content of the biogas.

A zero variant control was made of 3 replicates of inoculum without the test substrates. The standard variants were composed of 3 replicates with hay and 3 replicates with concentrate feed. The standards were purchased from the Institute for Animal Nutrition of the University of Hohenheim, Germany, and served as activity control by the in-vitro gas production technique for animal feeds. The gas yields of standards were evaluated in numerous trials to validate the expected values.

### Example 2

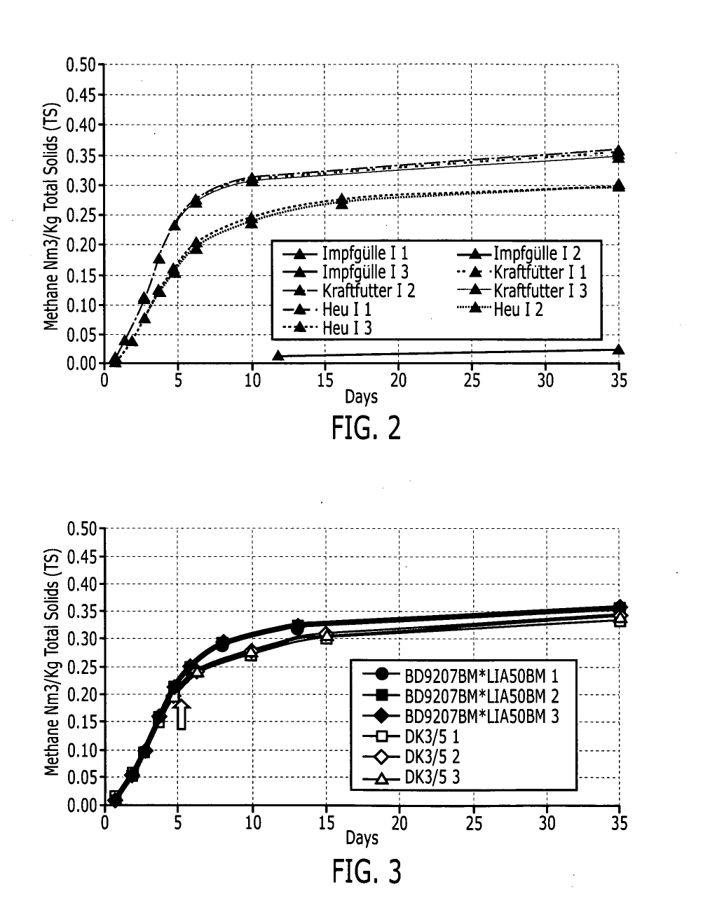

Methane yield potential measurements for various BMR corn hybrids for biogas production. Methods outlined in Example 1 were used to determine methane yield potential based on the methane yields of various BMR corn hybrid substrates. An example for the progression of methane production from the inoculums, substrate, hay and concentrate feed, is shown in FIG. 2. The course of the methane production did not show any substantial fluctuations. The inoculum was characterized as a low methane producing inoculum to minimize proportion of methane produced by the inoculum itself. As illustrated in FIG. 2, the methane production from the substrates started immediately after the experiment commenced, thereby indicating that the bacterial activity of the inoculum was sufficiently high.

The substrate specific methane yield of hay and concentrate feed standards as averages of three replicates each were 298 and 343 liter methane per kilogram of volatile solids (norm liter (NL) CH₄/Kg of Total Solids (TS)), respectively. Correspondingly, the relative standard deviations of variants were of 0.3% and 1.4%. The average values from the numerous previous trials with the same substrates were of 300 NL CH₄/Kg of TS for hay and of 340 NL CH₄/Kg of TS for concentrate feed.

FIG. 3 shows the evolution of the methane production of three replicates from two selected maize hybrids. The methane production also started after the trial began. A clear difference in methane yield was noticeable between the two maize varieties at about 5 days after the experiment was started. Until about day 5, the conversion of the energy stored inside of the crop into methane occurred more regularly with the BMR maize hybrid BD9207×LIA5OBM than with the conventional maize hybrid DK 3/5.

There were only a few perturbations within the digestion trials overall. The crop samples BD9207BM×LIA03BM and BE1146BMR×PIA03BM had relative standard deviations above 3.5% and these crop samples were in an independent experiment.

Table 1 presents a summary of the results obtained with various BMR and non-BMR hybrid plants for their methane yield potential. Relative standard deviations of all the variants were under 3.5%. The methane content in biogas of the different maize varieties had a small variation range, from 50.9% for F7026BM×3633 BMR to about 52.4% for BE1146BMR×3633 BMR. For calculation of methane yields per unit of cultivated plant material, the substrate specific methane yields of the crops is multiplied by the volatile solids yield per unit of cultivated surface, determined at the harvest of the material.]

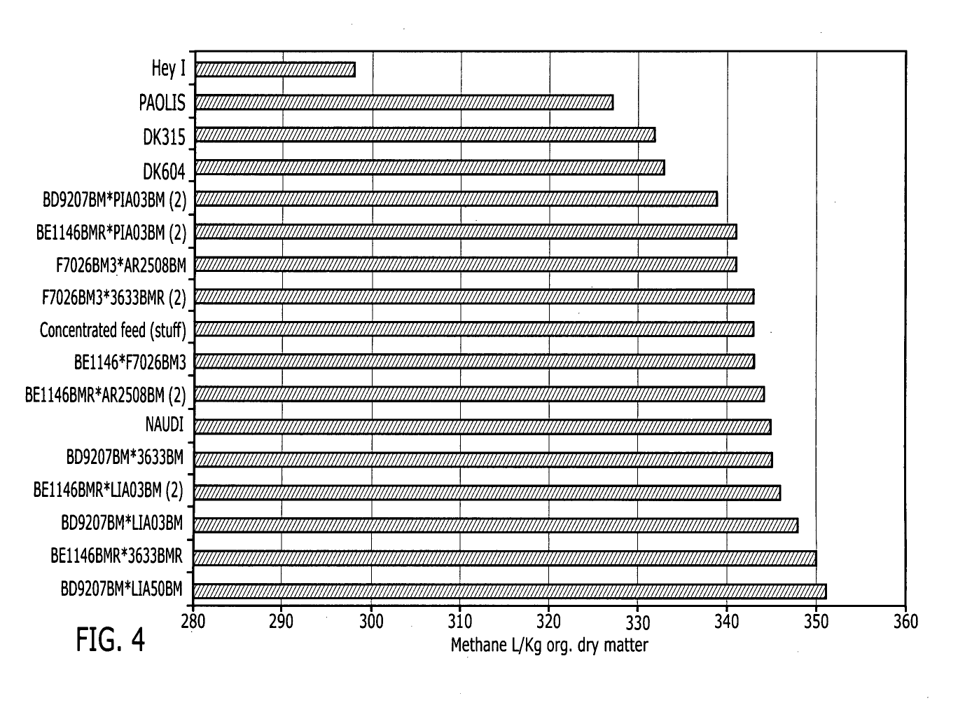

FIG. 4 is a bar graph showing methane production per kg. of organic dry matter for BMR and non-BMR maize hybrid plants. Some of the BMR varieties such as, for example, BD9207BM*PIA03BM, BE 1146BMR*L1A03BM, BE1146BMR*3633BMR, AND BR9207BM*L1A50BM show a higher average methane yield of about 345-352 liters of methane/kg organic dry matter compared to some of the non-BMR hybrids (DK 3/5; DK 604).

### Example 3

Production of biogas from plant material including cultivated plant biomass that includes BMR maize hybrids. As disclosed herein, methane yields from BMR hybrid corn plants were significantly higher. Dried silage material from cultivated BMR corn plants is obtained, processed for particulate size, if necessary and fed into an anaerobic digester of a suitable size. Anaerobic digesters to process plant material from BMR corn plants are obtained commercially or designed and built using knowledge available to a skilled artisan. Methods to determine methane yield potential from BMR corn plants are disclosed herein to provide yield coefficients for designing large-scale reactors. Anaerobic digesters for organic material including animal wastes and industrial wastes are available.

Plant material harvested from BMR corn plants is fed into a reactor of suitable scale. Microbial inoculum is added and appropriate temperature, pH, and volume are maintained during the anaerobic digestion step. This anaerobic digestion produces a “biogas” which includes mainly methane and carbon dioxide, but which also contains quantities of hydrogen sulfide and water. Depending on the sulfur content of the biomass, it may be necessary to remove the hydrogen sulfide, because H₂S is a highly corrosive compound. Methane produced from the digestion is often tested for purity and if further cleaning is necessary, appropriate purification steps are performed. Cleaned methane is then stored for further use or immediately used to generate electricity, heat or other forms of readily available energy.

### Example 4

Production of fertilizer material from anaerobic digestion of BMR corn plants. As explained in Example 3, biogas, which comprises mainly methane and carbon dioxide is removed for further energy-related uses. The left-over material is often rich in ammonium, nitrates, phosphates, and other minerals that were originally present in the plant material. The left-over material, along with the microbial mass, can be directly used as a fertilizer or a fertilizer supplement for farm plants and home plants. The left-over material can also be further processed. Because the anaerobic microbes are slow growing, the microbial mass may not be a significant concern considering the total biomass used for biogas generation. The fertilizer material may also be dried further, pulverized, and supplemented with other nutrients and minerals as needed.

### Example 5

Operation and modeling of anaerobic digesters. Anaerobic digestion of organic material including cultivated plant material involves a multitude of reaction steps. The insoluble organics undergo a liquefaction reaction by the extracellular enzymes present in the microbial inoculum to result in a substrate fraction that is substantially soluble. The soluble organic material is then acted upon by a broad spectrum of acid producing microbes. The bacteria produce proteolytic, lipolytic, and several cellulolytic enzymes. The solubilization reaction proceeds fast enough to prevent this step from becoming a rate limiting step.

The next step in the digestion process involves bacterial synthesis of short-chain fatty and volatile acids which also occur at a relatively rapid rate. Bacteria including facultative heterotrophs carry out this reaction, which function at a pH range of 4.0-6.5. Major product of this step is acetic acid, while propionic and butyric acid are also produced. There are over 50 species of methanogens, divided into three classes—the Methanobacteria, Methanococci, and Methanopyri.

About 70% of the methane that is produced from the biomass is generated from acetic acid during anaerobic digestion. The methane generation phase generally involves strict anaerobes and a pH range of about 7.0-7.8 is optimal, although they thrive in a wide range of environments. The methane generation step (conversion of volatile acids to CH₄ and CO₂) is usually the rate-limiting step depending on the content and nature of the biomass used.

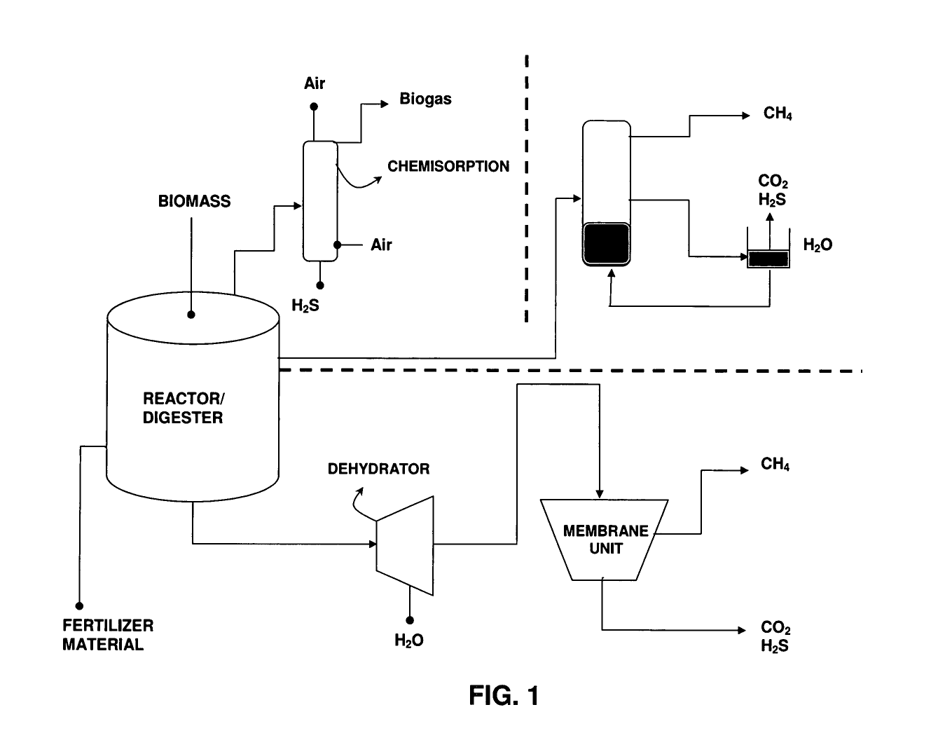

In an anaerobic digester as illustrated in FIG. 1, mixing is usually provided to prevent high local concentrations of acids, which may alter the local pH. Several purification methods exist to remove unwanted gases and other impurities. For example, as illustrated in FIG. 1, hydrogen sulfide is removed from the biogas by a process known as chemisorption, usually using iron oxide or zinc oxide. Sometimes, carbon dioxide scrubbing column is used to increase the yield of methane in the resultant biogas. In another illustrated method in FIG. 1, water absorption removes the impurities including hydrogen sulfide. In yet another process shown in FIG. 1, membrane separation in the membrane element follows removal of excess water in the dehydrator. On one side of the membrane, methane gas is yielded and on the other, carbon dioxide and hydrogen sulfide are removed. Generally, anaerobic digesters as shown in FIG. 1 are equipped with a variety of features including gas blow off pipes, vacuum/pressure release valves, flame traps, circulating water and expansion chamber, and heat exchangers.

Generally, to maintain a suitable reaction environment for both the acid forming bacteria and the methane producing bacteria, digesters are operated at a pH of about 7. However, depending on the nature of the biomass, for example, biomass of BMR plants that do not have as much lignin content as other plant biomass, the pH can be altered to favor the methane producing bacteria more than the acid formers. Alternatively, pH can be adjusted progressively as the digestion shifts from acid hydrolysis to methane production.

Depending on the nature of the inoculum, the temperature can range from about 55° F. to about 100° F. for mesophilic bacteria and up to 150° F. for thermophilic bacteria. However, to minimize energy costs to maintain the ambient temperature in the reactor, digestion temperatures are often in the mesophilic range. Based on the nature of the plant material, the inoculum, and the operating temperature, the residence time of the plant material in the reactor may range from about 10 days to about 40 days. When a high methane yielding plant variety such as, for example, BMR corn-based plant material is used, the residence time is shorter and the methane yield is higher per unit organic dry weight.

An anaerobic digester is essentially a three phase system. The model assumes a gas phase in contact but not in equilibrium with the liquid phase. Gas phase is assumed to obey the ideal gas law. Methane is assumed to be water insoluble and directly transferable to the gas phase, whereas the generated CO₂ partly dissolves in the liquid phase giving carbonic acid, which depending on the pH is dissociated giving bicarbonate and carbonate ions, and partly escapes to the gas phase, see Graef and Andrews, (1974), Stability and control of anaerobic digestion. Journal WPCF, 46, 667-682, incorporated herein by reference; Graef and Andrews (1974). Mathematical modeling and control of anaerobic digestion. CEP Symp. Ser. 136:101-127.

Graef and Andrews (1974) describe a Monad expression to calculate the rate of methane production:

Q_{CH4}=V/ρg*Y_{CH4/x}*μx
Where Q_{CH4} is the rate of methane formation; V is the volume of the reactor; Y_{CH4/x} is the yield coefficient for methane; μ is the growth rate of the microbial inoculum; and x is the cell mass.

Several models derived from Graef and Andrews (1974) have been adapted for a variety of anaerobic digestions to produce biogas from a variety of sources.

TABLE 1
Summary of the results

| | | | | | | Relative |
|---|---|---|---|---|---|---|
| | | | | | Average of | standard |
| | | | Average of | Substrate specific | specific | deviation (% |
| | | Methane content | methane | methane yield (Nm3 | methane | of average |
| | | (% of gas volume) | content | CH4/kg VS | yield | value) |
| 10 | BD9207BM*LIA03BM 1 | 52 | 51.7 | 0.351 | 0.348 | 0.8 |
| 11 | BD9207BM*LIA03BM 2 | 52 | | 0.347 | | |
| 12 | BD9207BM*LIA03BM 3 | 52 | | 0.346 | | |
| 13 | BD9207BM*LIA50BM 1 | 52 | 51.8 | 0.349 | 0.351 | 0.4 |
| 14 | BD9207BM*LIA50BM 2 | 52 | | 0.351 | | |
| 15 | BD9207BM*LIA50BM 3 | 52 | | 0.352 | | |
| 16 | BD9207BM*PIA03BM 1 | 52 | 51.9 | 0.347 | 0.339 | 3.5 |
| 17 | BD9207BM*PIA03BM 2 | 52 | | 0.330 | | |
| 18 | BD9207BM*PIA03BM 3 | 52 | | rejected value | | |
| 19 | BD9207BM*3633BM 1 | 52 | 52.2 | 0.353 | 0.352 | 0.5 |
| 20 | BD9207BM*3633BM 2 | 52 | | 0.350 | | |
| 21 | BD9207BM*3633BM 3 | 52 | | rejected value | | |
| 22 | BE1146BMR*AR2508BM 1 | 52 | 51.7 | 0.346 | 0.344 | 0.7 |
| 23 | BE1146BMR*AR2508BM 2 | 52 | | 0.342 | | |
| 24 | BE1146BMR*AR2508BM 3 | 51 | | rejected value | | |
| 25 | BE1146BMR*LIA03BM 1 | 52 | 51.8 | 0.345 | 0.346 | 0.5 |
| 26 | BE1146BMR*LIA03BM 2 | 51 | | rejected value | | |
| 27 | BE1146BMR*LIA03BM 3 | 52 | | 0.348 | | |
| 28 | BE1146BMR*PIA03BM 1 | 52 | 51.5 | 0.343 | 0.341 | 1.0 |
| 29 | BE1146BMR*PIA03BM 2 | 51 | | rejected value | | |
| 30 | BE1146BMR*PIA03BM 3 | 51 | | 0.338 | | |
| 31 | BE1146BMR*3633BMR 1 | 53 | 52.4 | 0.348 | 0.350 | 0.5 |
| 32 | BE1146BMR*3633BMR 2 | 52 | | 0.350 | | |
| 33 | BE1146BMR*3633BMR 3 | 52 | | 0.352 | | |
| 34 | BE1146*F7026BM3 1 | 51 | 51.0 | 0.344 | 0.343 | 1.4 |
| 35 | BE1146*F7026BM3 2 | 51 | | 0.338 | | |
| 36 | BE1146*F7026BM3 3 | 51 | | 0.347 | | |
| 37 | DK3/5 1 | 51 | 51.4 | 0.329 | 0.332 | 1.6 |
| 38 | DK3/5 2 | 52 | | 0.328 | | |
| 39 | DK3/5 3 | 51 | | 0.338 | | |
| 40 | DK604 1 | 52 | 51.7 | 0.335 | 0.333 | 1.9 |
| 41 | DK604 2 | 52 | | 0.325 | | |
| 42 | DK604 3 | 52 | | 0.338 | | |
| 43 | F7026BM3*AR2508BM 1 | 51 | 51.2 | 0.345 | 0.341 | 1.1 |
| 44 | F7026BM3*AR2508BM 2 | 51 | | 0.341 | | |
| 45 | F7026BM3*AR2508BM 3 | 51 | | 0.338 | | |
| 46 | F7026BM3*3633BMR 1 | 51 | 50.9 | rejected value | 0.343 | 0.2 |
| 47 | F7026BM3*3633BMR 2 | 51 | | 0.342 | | |
| 48 | F7026BM3*3633BMR 3 | 51 | | 0.343 | | |
| 49 | NAUDI 1 | 52 | 51.7 | 0.342 | 0.345 | 0.8 |
| 50 | NAUDI 2 | 52 | | 0.347 | | |
| 51 | NAUDI 3 | 52 | | 0.346 | | |
| 52 | PAOLIS 1 | 52 | 51.6 | 0.325 | 0.327 | 0.6 |
| 53 | PAOLIS 2 | 51 | | 0.327 | | |
| 54 | PAOLIS 3 | 52 | | 0.329 | | |
| 55 | 11084BMR*3633BMR 1 | 51 | 51.6 | 0.327 | 0.323 | 1.4 |
| 56 | 11084BMR*3633BMR 2 | 52 | | 0.319 | | |
| 57 | 11084BMR*3633BMR 3 | 52 | | 0.321 | | |

1. A method of producing a biogas, the method comprising the steps of: providing a plant material characterized by a reduced lignin content relative to a wild type plant material and anaerobically digesting the plant material with a microbial inoculum to produce the biogas.

2. The method of claim 1, wherein the plant material characterized by reduced lignin content relative to a wild type plant material comprises a brown midrib (BMR) corn or BMR sorghum.

3. The method of claim 1, wherein the biogas comprises methane.

4. The method of claim 3, wherein the methane is produced at a concentration of at least about 340 liters of methane per kilogram of dry organic matter.

5. The method of claim 1, wherein the biogas comprises about 50% methane by volume.

6. The method of claim 1, wherein the microbial inoculum comprises acid forming bacteria and methane producing bacteria.

7. The method of claim 1, wherein the plant material comprises a silage or a plant stover.

8. The method of claim 1, wherein the plant material is a feed concentrate.

9. The method of claim 1, wherein the digesting step is performed at a temperature of about 55° F. to about 100° F. and at a pH of about 7 to about 8.

10. The method of claim 1, wherein the digesting step is performed in an anaerobic reactor.

11. The method of claim 1, wherein the plant material is processed before performing the digesting step.

12. The method of claim 11, wherein the processing step includes a step selected from the group consisting of drying, milling, and acid hydrolysis.

13. A method for producing a digested substrate, the method comprising the steps of: providing a plant material characterized by reduced lignin content relative to a wild type plant material; anaerobically digesting the plant material in the presence of a microbial inoculum to produce a biogas; and, recovering the digested substrate.

14. The method of claim 13, wherein the recovered digested substrate is in a dried state.

15. The method of claim 13, wherein the recovered digested substrate is a sludge.

16. A method for determining a methane yield potential of a plant material characterized by reduced lignin content relative to a wild type plant material, the method comprising:
(a) obtaining a substrate sample from the plant material characterized by reduced lignin content;
(b) allowing the substrate sample to contact a microbial inoculum in a container under conditions such that the substrate sample produces a biogas; and
(c) determining the methane yield potential of the plant material.

17. The method of claim 16, wherein the plant material is a BMR plant material.

18. The method of claim 17, wherein the BMR plant material is selected from the group consisting of BMR corn and BMR sorghum.
